(19) United States
(12) Patent Application Publication
(10) Pub. No.: US 2026/0157614 A1
(43) Pub. Date: Jun. 11, 2026

(54) FLEXING STRUCTURE FOR A MEDICAL DEVICE, WITH REINFORCED HOLDING TOGETHER OF ARTICULATED VERTEBRAE

(71) Applicant: AXESS VISION TECHNOLOGY, JOUE-LES-TOURS (FR)

(72) Inventors: Philippe LE ROUX, TOURS (FR); Luka LECOUTEY, ROCHECORBON (FR)

(21) Appl. No.: 18/706,964

(22) PCT Filed: Nov. 2, 2022
(86) PCT No.: PCT/FR2022/052062

§ 371 Date: May 2, 2024

(30) Foreign Application Priority Data
Nov. 3, 2021 (FR) 21 11670

(51) Int. Cl. A61B 1/005; A61B 1/00

(52) U.S. Cl. CPC A61B 1/0055; A61B 1/0011; A61B 1/0057

(57) ABSTRACT

A bending structure for an insertion tube of a medical device is disclosed. The bending structure includes a tubular vertebrae and at least one actuation wire extending in a diametrical bending plane of the structure, the bending structure having a system for retaining the tubular vertebrae having at least one lug extending projecting from the male cut-out areas and engaged in a slot in the female cut-out areas and having two rotation limiting surfaces, and a pair of hooks extending projecting from the tubular vertebrae, outside the diametrical bending plane, the hooks each being engaged in a housing arranged in a neighboring tubular vertebra and having a retaining stop on which the hook rests.

### TECHNICAL FIELD

The present disclosure concerns the technical field of medical devices in the general sense allowing access to the interior of a body such as a cavity or a canal, for example, and it more precisely relates to medical devices of the catheter type and preferably, medical devices of the endoscope type.

The object of the present disclosure finds a particularly advantageous application for reusable or single-use endoscopes.

The present disclosure more precisely concerns the bending structure making it possible to guide the distal head of such medical devices of the catheter or endoscope type, this distal head being adapted to ensure multiple functions such as viewing, fluid supply, fluid suction, instrument supply, sample taking or surgical operations, for example.

The medical device of the catheter or endoscope type equipped with the bending structure of the distal head according to the present disclosure finds particularly advantageous applications for allowing access to the internal surface of a hollow organ, a cavity or a natural or artificial conduit of the human body for the purpose of carrying out various operations for therapeutic, surgical or diagnostic purposes. For example, the medical device of the catheter or endoscope type according to the present disclosure can be used in the area of the urinary tract, the gastrointestinal tract, the respiratory system, the cardiovascular system, the trachea, the sinus cavity, the female reproductive system, the abdominal cavity or any other part of the human body to be explored by a natural or artificial means.

### BACKGROUND

Generally, a medical endoscope, as described for example by patent application WO 2014/106510, has an operating handle to which an insertion tube is attached. This tube has a distal head equipped with an optical vision system making it possible to illuminate and examine the organ, cavity or conduit of the human body. Upstream of this distal head, the insertion tube has a bending structure or articulated part formed of articulated tubular vertebrae making it possible to guide the distal head using one or more actuation wires mounted inside the insertion tube. Each actuation wire has a first end fixed to the distal head and a second end on which a control mechanism fitted to the handle acts to cause the wires to slide and, consequently, cause this articulated part to fold in order to guide the distal head.

The manufacture of this articulated part requires the assembly of the vertebrae, leading to difficulty in assembly and a high manufacturing cost. This manufacturing difficulty is even more problematic since, in many applications, there appears the need to miniaturize such an endoscope to allow its passage through an access route of reduced diameter. This miniaturization must take into account the need to mount, inside the insertion tube, various equipment designed to allow different functions to be carried out such as fluid supply, fluid suction, instrument supply, sample taking or surgical operations, or routing vision system connections.

To satisfy this need, the state of the art, for example U.S. Pat. No. 10,687,695, has proposed producing the articulated part or the bending structure, using a tube cut by cutting lines of a beam of energy to produce tubular vertebrae nested within each other by cut-out areas forming pivots of rotation along axes of rotation. Notches are created between the tubular vertebrae, cut into the tube to form bending zones for the tubular vertebrae in order to obtain the bending of the bending structure in at least one bending plane perpendicular to the axes of rotation. Such a bending structure has a low manufacturing cost while being able to have a reduced cross segment.

This patent proposes creating sliding surfaces on the tubular vertebrae to resist forces perpendicular to the axes of rotation in order to prevent the vertebrae from disengaging. In practice, this solution is complex to manufacture, especially for tubular vertebrae with a small diameter, typically less than 10 mm.

Patent application WO2020135364 describes a bending structure for an endoscope having a series of tubular vertebrae nested within each other by male cut-out areas and female cut-out areas forming rotation pivots. This bending structure has a retaining system limiting the rotation of the tubular vertebrae between each other between two opposite extreme positions. This retaining system has, for each pivot, two bulges engaged in grooves and extending two arms extending on either side of the pivot, and projecting from the cut out edge of a tubular vertebra. This bending structure has low torsional strength given the significant cuts made. In addition, this architecture leads to a pivot of limited dimensions that does not make it possible to obtain good radial hold, which entails a significant risk of disengagement of the tubular vertebrae.

U.S. Pat. No. 10,816,118 describes a bending structure for an endoscope having a series of tubular vertebrae nested within each other by male cut-out areas and female cut-out areas forming rotation pivots. This bending structure also has a system for retaining the tubular vertebrae together between two opposite extreme positions. This support system has two T-shaped arms extending on either side of the tubular vertebrae in the bending plane of the structure. These two arms extend projecting from a tubular vertebra and are each engaged in a recess provided in a neighbouring tubular vertebra. Such a retaining system makes it possible to reinforce the strength of the structure in a direction perpendicular to the bending plane of the structure. However, the position of this retaining system in the bending plane leads to an interaction between the arms of the retaining system and the control for the bending of the structure, leading to an alteration of the bending performance of the structure.

A bending structure for an insertion tube of a medical device is also known from the document JP 2015 047453, having tubular vertebrae articulated together by male cut-out areas and female cut-out areas forming rotation pivots. Each female cut-out area is delimited by two annular segments partially surrounding a male cut-out area to abut on the narrowed neck of the male part in the extreme bending position. According to a variant embodiment, this bending structure has, on either side of the insertion tube, a pair of flexible tabs arranged on either side of the diametrical bending plane extending projecting from a tubular vertebra to cooperate with one of the notches in a series arranged on a neighbouring tubular vertebra. The cooperation of the tabs with the notches makes it possible to define an articulation position between the tubular vertebrae. However, such a structure does not ensure that the tubular vertebrae are locked in extreme positions of articulation. Moreover, the extreme rotational positions obtained by abutting the annular segments on the narrowed neck of the male segments leads to a loss of rotational guidance between the pivots or to a limitation of the bending angle.

Document CN112294237 describes a bending structure having tubular vertebrae articulated together by male cut-out areas and female cut-out areas forming rotation pivots. A narrowed neck extends from each male cut-out area, provided at its end with an annular segment laterally having two rotation limiting surfaces intended to cooperate with stops presented by a recess in which the annular segment is mounted. The creation of such recesses weakens the bending structure.

Document US2020/0237188 describes a bending structure having tubular vertebrae articulated together by male cut-out areas and female cut-out areas forming rotation pivots. Each male cut-out area is delimited by an annular-shaped housing into which an annular segment, abutting to limit pivoting, is inserted. The cuts made in the tubular vertebrae to create the recesses weaken the bending structure.

Document CN109497915 describes a bending structure having tubular vertebrae articulated together by male cut-out areas and female cut-out areas forming rotation pivots. According to an exemplary embodiment, annular segments extend on either side of each male cut-out area to cooperate with annular-shaped recesses. The cuts made in the tubular vertebrae to create the recesses weaken the bending structure. According to another exemplary embodiment, the female cut-out areas are arranged to present, on either side of the pivot, tabs engaged in recesses. These recesses arranged on either side of the pivot also weaken the bending structure.

### SUMMARY

The present disclosure aims to remedy the disadvantages of the state of the art by proposing a bending structure for a medical device of the catheter or endoscope type, designed to ensure that the tubular vertebrae are effectively held together regardless of the direction of the forces exerted on the structure, without altering the bending performance of the structure.

To achieve such an objective, the object of the present disclosure concerns a bending structure for an insertion tube of a medical device, having tubular vertebrae with a proximal tubular vertebra and a distal tubular vertebra and at least one actuation wire extending in a diametrical bending plane of the structure, the bending structure having a tube with a longitudinal axis, cut by cutting lines of an energy beam to produce tubular vertebrae nested in one another by diametrically opposed male cut-out areas and diametrically opposed female cut-out areas forming pivots of rotation along axes of rotation located in a plane of symmetry, the cutting lines being arranged to delimit, between the tubular vertebrae, notches cut in the tube to form bending zones for the tubular vertebrae in order to obtain the bending of the bending structure in the diametrical bending plane perpendicular to the axes of rotation, the bending structure having a system for retaining the tubular vertebrae together between two opposite extreme positions. In accordance with the present disclosure, the retaining system has:
at least one lug extending projecting from the male cut-out areas and engaged in a slot in the female cut-out areas and having two rotation limiting surfaces located on either side of the plane of symmetry,
a pair of hooks extending projecting from the tubular vertebrae, outside the diametrical bending plane and in orthogonal symmetry relative to the longitudinal axis, each hook having an L shape with a rectilinear shank curved at its end, on one side to present a heel delimiting, opposite the end of the shank, a retaining surface, the hooks each being engaged in a housing arranged in a neighbouring tubular vertebra opening in an outer edge of said neighbouring tubular vertebra by a narrowed part for guiding the rectilinear shank bordered by a retaining stop on which the hook comes to rest by its heel in the two extreme positions.

Preferably, for each hook, the retaining stop has a flat wall.

According to one advantageous characteristic of the present disclosure, the retaining system is arranged in such a way that for each extreme position between two neighbouring tubular vertebrae, on the one hand, the two lugs of two diametrically opposed male cut-out areas are supported on the limiting surfaces of slots located on one side of the plane of symmetry located opposite the bending direction and, on the other hand, the hook located on the side of this plane of symmetry, i.e., supported on the retaining stop.

According to an exemplary embodiment, two lugs extend projecting from each of the male cut-out areas and are each engaged in a slot in the female cut-out areas and have a first bearing surface and a second bearing surface intended to cooperate with a rotation limiting surface.

According to an advantageous embodiment, each lug has at least one bearing surface not passing through the axis of rotation and intended to cooperate, in an extreme position, with a rotation limiting surface extending along a plane not passing through the axis of rotation, so as to nest one inside the other.

Advantageously, the two surfaces limiting the rotation of each lug extend projecting from the male cut-out areas and to the outer edge of the lug.

According to another advantageous characteristic of embodiment, each male cut-out area has a connecting neck with the part forming a rotation pivot.

Preferably, each female cut-out area is arranged to connect with an outer edge of the tubular vertebra, using non-pointed reinforcing heels extending on either side of the part forming a rotation pivot and intended to engage in the connecting neck.

According to one embodiment, the retaining system has, for at least one tubular vertebra, two pairs of tongues arranged symmetrically on either side and outside the diametrical bending plane, each being engaged in a groove provided in a neighbouring tubular vertebra.

For example, the tube has a diameter less than or equal to 10 mm.

Advantageously, the tube has a wall thickness comprised between 0.05 and 1.5 mm.

Another object of the present disclosure is to propose a tube for insertion of a medical device of the endoscope or catheter type having a bending structure according to the present disclosure, fixed to the tube at its proximal end and to a distal head at its distal end.

Another object of the present disclosure is to propose a medical device having an operating handle equipped at its distal part with an insertion tube according to the present disclosure.

### BRIEF DESCRIPTION OF THE DRAWINGS

FIG. 1 is a schematic general view of a medical device of the catheter type in the general sense equipped with a bending structure according to the present disclosure.

FIG. 2 is a top view more particularly showing a preferred exemplary embodiment of a bending structure according to the present disclosure, in the non-articulated position.

FIG. 3 is a top view showing more particularly a preferred exemplary embodiment of a bending structure according to the present disclosure, in the articulated position.

FIG. 4 is a side perspective view showing part of a bending structure according to the present disclosure, in the articulated position.

FIG. 5 is a top view showing in detail a part of a bending structure according to the present disclosure, in the articulated position.

FIG. 6 is a perspective view in longitudinal segment showing part of the interior of a bending structure according to the present disclosure.

FIG. 7 is a side view of a bending structure according to the present disclosure, in a non-articulated position, with cut-out areas.

FIG. 8 is a view showing the end of a tubular vertebra making up a bending structure according to the present disclosure.

FIG. 9 is a perspective view showing another embodiment of a retaining system forming part of a bending structure according to the present disclosure.

FIG. 10 is a top view showing another exemplary embodiment of a retaining system of a bending structure according to the present disclosure, in non-articulated position.

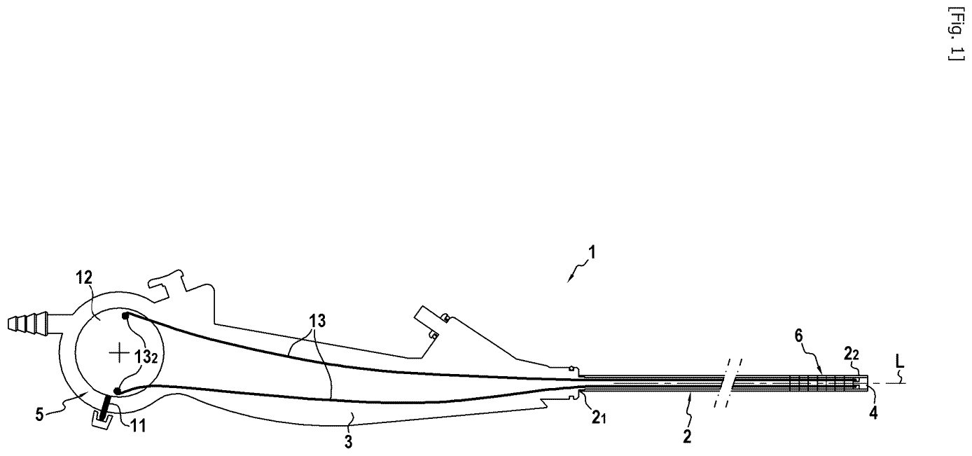

### DETAILED DESCRIPTION

The object of the present disclosure concerns a medical device 1 of the endoscope or catheter type 1 in the general sense designed to access the interior of a body such as a cavity or a canal, for example. Conventionally, a medical device 1 of the endoscope or catheter type has an insertion tube 2 having on one side, a proximal part 21 connected to an operating handle 3 and on the opposite side, a distal part 22 which is equipped with a distal head 4. The insertion tube 2 is fixed temporarily or permanently on the operating handle 3. This insertion tube 2, which has a greater or lesser length and flexibility, is intended to be introduced into a natural or artificial access route with a view to carrying out various operations or functions for therapeutic, surgical or diagnostic purposes. The insertion tube 2 is made of a semi-rigid material and has a length adapted to the length of the conduit to be inspected and which can be between 5 cm and 2 m. The insertion tube 2 has various cross-segment shapes such as square, oval or circular. This insertion tube 2, which is in contact with human tissues or organs or with medical equipment (trocars or probes), is essentially for single or multiple use by a patient or even for reusable use after decontamination, disinfection or sterilization.

According to a preferred embodiment illustrated in the figures, the medical device 1 according to the present disclosure is an endoscope having a vision system capable of illuminating and returning an image of the distal part of the insertion tube 2. The endoscope thus has a vision system mounted inside the operating handle 3 and penetrating inside the insertion tube 2 up to the distal head 4. Likewise, the medical device 1 also has, inside the insertion tube 2, an operating or working channel extending from the operating handle 3 to the distal head 4 to allow the supply of various tools and/or fluids and/or to allow the suction of fluids.

Conventionally, the medical device 1 also has a control mechanism 5 making it possible to orient the distal head 4 relative to the longitudinal axis L of the insertion tube 2. For this purpose, the insertion tube 2 has, upstream of the distal head 4, a bending, folding or articulating structure 6 according to the present disclosure allowing the orientation of the distal head 4 relative to the longitudinal axis L of the insertion tube 2.

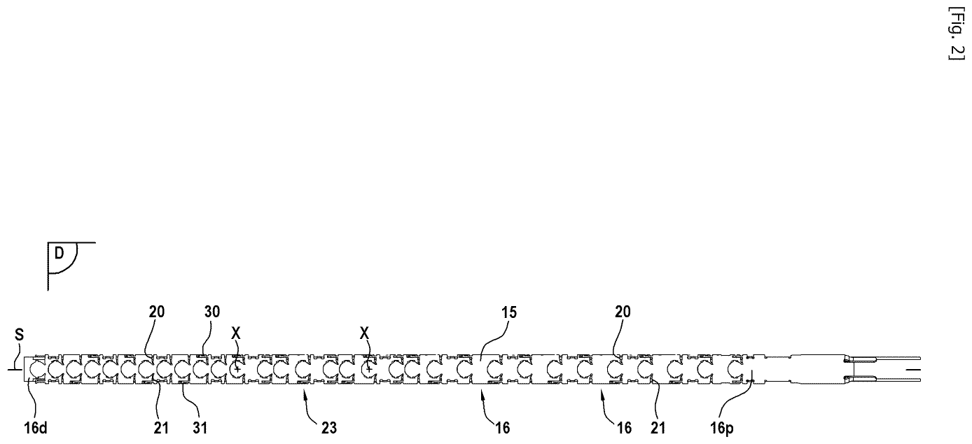

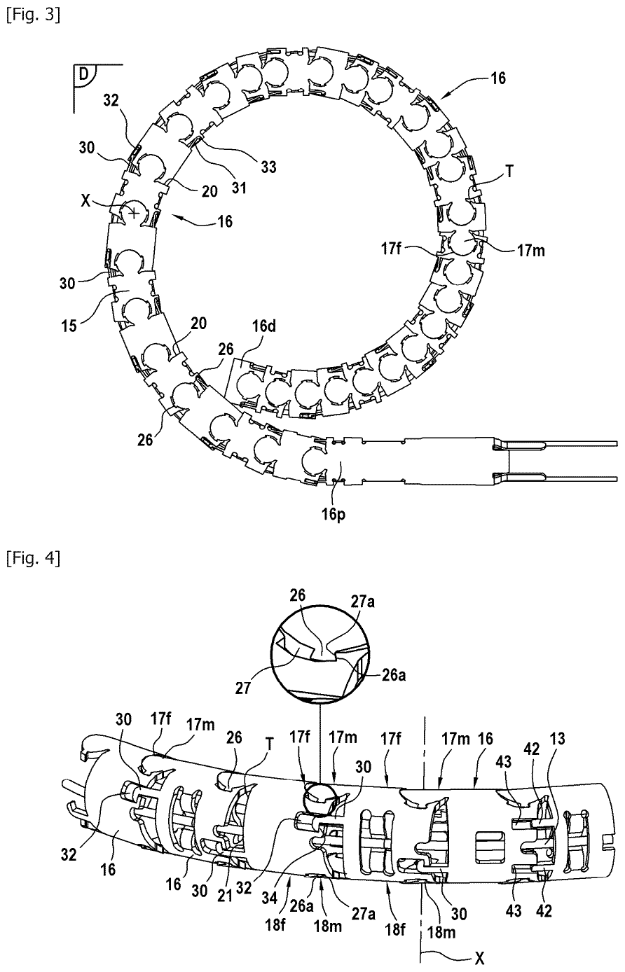

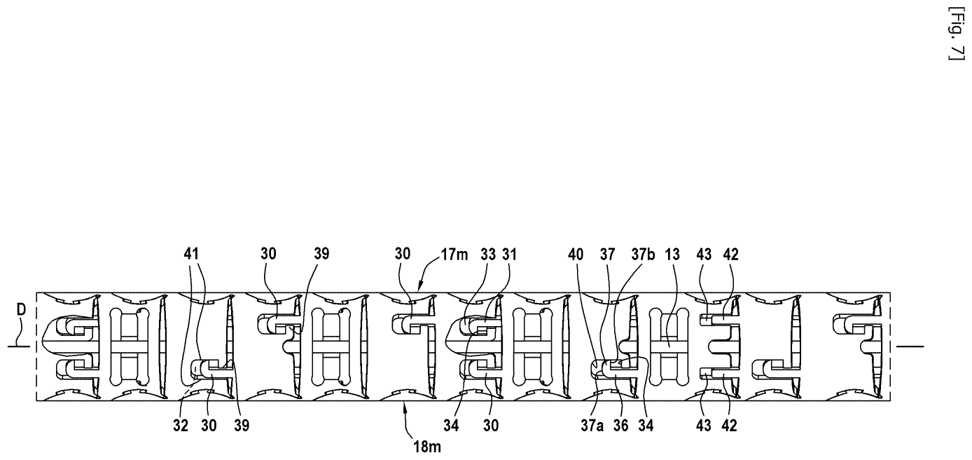

The control mechanism 5 can be made in any suitable manner so that the distal head 4 can be moved between a rest position in which the insertion tube 2, including the bending structure, is rectilinear (FIGS. 2 and 7) and an articulated position in which the bending structure 6 is curved (FIGS. 3, 4 and 5). As a non-limiting example, the control mechanism 5 may correspond to the control mechanism described in patent FR 3 047 887. For this purpose, the control mechanism 5 has a manual control lever 11 rotating at least one pivoting part such as a pulley 12 on which is fixed at least one actuation wire 13 mounted inside the insertion tube 2 to be fixed at the distal head 4. Typically, each actuation wire 13 is surrounded by a sheath over at least part of its length.

According to a preferred embodiment illustrated by the figures, the bending structure 6 has a tube 15 of longitudinal axis L, cut by cutting lines T coming from an energy beam to produce rings or tubular vertebrae 16 nested into one another by a first series of cut-out areas 17m, 17f forming pivots of rotation and by a second series of cut-out areas 18m, 18f also forming pivots of rotation. The cut-out areas 17m, 17f of the first series extend diametrically opposed to the cut-out areas 18m, 18f of the second series to form first axes of rotation X. Thus, two neighbouring tubular vertebrae 16 are adapted to pivot relatively between them around a first axis of rotation X passing through a rotation pivot 17m, 17f of the first series and through a rotation pivot 18m, 18f of the second series. By convention, it is considered that the tube 15 has, at rest, a plane of symmetry S in which the first axes of rotation X are located, extending parallel to each other.

Advantageously, each series of cut-out areas alternately has male cut-out areas 17m, 18m nested in female cut-out areas respectively 17f, 18f. Thus, a tubular vertebra 16 has two male cut-out areas 17m, 18m located diametrically opposite each other cooperating with two female cut-out areas 17f, 18f also arranged diametrically opposite in the adjacent tubular vertebra 16. Of course, the male cut-out shapes 17m, 18m are congruent or complementary to the female cut-out shapes 17f, 18f to allow relative rotation between two neighbouring tubular vertebrae 16 and interlocking between two neighbouring tubular vertebrae 16.

Each tubular vertebra 16 thus has, on the one hand, two male cut-out areas 17m, 18m extending in a superimposed manner from a first outer edge 16a of the tubular vertebra and, on the other hand, two female cut-out areas 17f, 18f arranged in a superimposed manner from a second outer edge 16b of the tubular vertebra, opposite the first outer edge 16a.

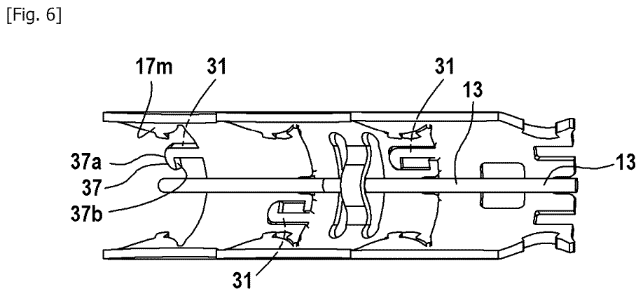

It should be understood that the cut-out areas 17m, 17f, 18m, 18f forming rotation pivots cooperate with each other through their thickness. In other words, a male cut-out area 17m, 18m is in contact or supported by its edge with the edge of the female cut-out area 17f, 18f. As is apparent from the exemplary embodiment illustrated in FIGS. 2 to 10, the male cut-out shapes 17m, 18m have, as a part forming a rotation pivot, portions of discs with an angular extent greater than 180° (for example between 200° and 300°) to be fitted into portions of rings or bearings.

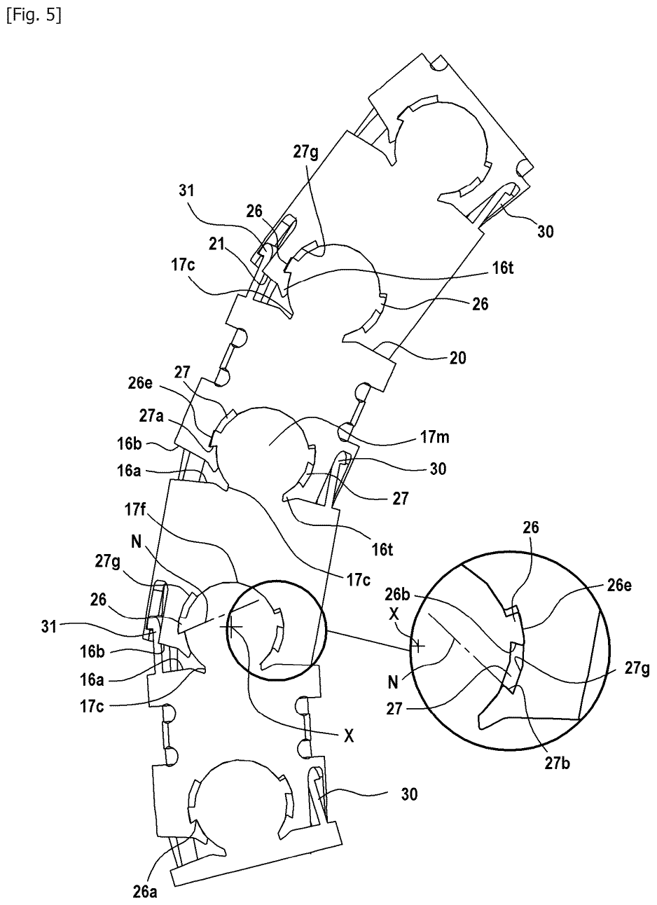

According to a preferred embodiment, each male cut-out area 17m, 18m has a connecting neck 17c with the disc portion forming a rotation pivot (FIG. 5). Preferably, this connecting neck 17c has a constant width extending over a height adapted to place the disk portion forming a rotation pivot in a spaced position relative to the first outer edge 16a. According to this exemplary embodiment, each female cut-out area 17f, 18f is arranged to connect with the second outer edge 16b of the tubular vertebra, using non-pointed reinforcing heels 16t extending on either side of the part forming a rotation pivot and intended to engage in the connecting neck 17c. It must be understood that each female cut-out area 17f, 18f does not connect with the second outer edge 16b of the tubular vertebra, with a pointed area to prevent this area from being blunted during maximum bending of the structure. These reinforcing heels 16t thus prevent the tubular vertebrae from disengaging during repeated bending stresses.

It should be understood that the bending structure 6 is obtained from a single tube 15 extending along a rectilinear longitudinal axis L and having a preferably circular cross segment. The tube 15 has a thickness designed to be able to be cut over its entire thickness by an energy beam of all known types, such as a plasma beam, a water jet or, preferably, a laser beam. For example, tube 15 is cut by CO2 or YAG laser. The tube 15 has a wall thickness comprised between 0.05 and 1.5 mm. The tube 15 has a diameter preferably less than or equal to 10 mm and, for example, comprised between 1 mm and 10 mm.

Likewise, the tube 15 is made of a material suitable for being cut by an energy beam while having the mechanical characteristics of bending and mechanical strength necessary for the bending structure 6. For example, tube 15 is made of stainless steel.

As shown in FIGS. 2 and 3, the cutting lines T are arranged to form a series of tubular vertebrae 16 in the tube, juxtaposed to each other, with a so-called distal tubular vertebra 16d and a so-called proximal tubular vertebra 16p. The bending structure 6 thus has at its distal end, a distal tubular vertebra 16d intended to be fixed to the distal head 4 of the medical device and at its proximal end, a proximal tubular vertebra 16p intended to be fixed to the distal end of the insertion tube 2. For example, the bending structure 6 mounted between the tube 2 and the distal head 4 is inserted inside a protective sheath or envelope.

These tubular vertebrae 16 have a length taken along the longitudinal axis comprised, for example, between 2 mm and 15 mm.

As appears from the exemplary embodiment illustrated in FIGS. 2 to 10, cutting lines T are arranged in the tube 15 to make a first series of notches 20 and a second series of notches 21 cut in the tube by extending symmetrically on either side of the plane of symmetry S to form bending zones for the tubular vertebrae 16. In other words, the notches 20, 21 are arranged to allow the pivoting of the tubular vertebrae between them around the first axis of rotation X, along a diametrical bending plane D perpendicular to the first axis of rotation X. The tube structure 6 thus has, on one side of the first plane of symmetry S, the notches 20 of the first series separating the tubular vertebrae 16 two by two and, on the other side of the plane of symmetry S, the notches 21 of the second series separating the tubular vertebrae 16 two by two.

It appears from the preceding description that the notches 20, 21 are made in the tube 15 with material removal. Each notch 20, 21 between two adjacent tubular vertebrae 16 results from a cut designed to produce the first outer edge 16a of a tubular vertebra and the second outer edge 16b of the neighbouring tubular vertebra 16. When bending the bending structure 6, in a direction perpendicular to the axes of rotation X, the part of the first outer edges 16a of the tubular vertebrae delimited by this first series of notches 20 comes to abut in the extreme bending position against the part of the second outer edges 16b of the neighbouring tubular vertebrae 16 delimited by this first series of notches 20. Of course, during bending of the bending structure 6, in a direction opposite the direction perpendicular to the axes of rotation X, the part of the outer edges 16a of the tubular vertebrae delimited by the second series of notches 21 comes to abut in the extreme bending in the extreme bending position against the part of the second outer edges 16b of the neighbouring tubular vertebrae 16 delimited by this second series of notches 21.

The notches 20 of the first series are made according to an angular extension range which is identical or variable to allow the adjustment of the pitch of orientation of the tubular vertebrae between them, that is to say the shape of the bending structure. Likewise, the notches 21 of the second series are made according to an identical or different angular extension range. For example, the notches 20 of the first series and the notches 21 of the second series are made according to an identical angular extension range. Advantageously, the notches 20 of the first series and the notches 21 of the second series are centred relative to the diametrical plane D passing through the diameter of the tube and perpendicular to the plane of symmetry S.

The notches 20 of the first series are made with a cutout that may or may not be identical in size and shape. Likewise, the notches 21 of the second series are made with a cutout that may or may not be identical in size and shape. Advantageously, the notches 20 of the first series and the notches 21 of the second series are made with a cutout identical in size and shape. The notches 20 of the first series and the notches 21 of the second series are made by cutouts of oblong or tapered shape, adapted to allow relative pivoting between two adjacent tubular vertebrae 16 along the first axes of rotation X.

In the exemplary embodiments illustrated in FIGS. 2 to 10, the tube structure 6 has a first series of notches 20 and a second series of notches 21 to obtain the bending of the bending structure in both ways of the direction perpendicular to the first axis of rotation X. Of course, a tube structure can be created having a single series of notches in the case where the tube structure 6 is provided with a single actuation wire 13 to obtain the bending of the bending structure in a single way of the direction perpendicular to the first axis of rotation X.

In the exemplary embodiments illustrated in FIGS. 2 to 10, the tubular vertebrae 16 are articulated along only the first axis of rotation X in order to obtain the bending of the bending structure in a perpendicular plane, i.e., the diametrical bending plane D. According to this example, the tube structure 6 has two actuation wires 13 located in the diametrical bending plane D. As appears in the drawings, the two actuation wires 13 are mounted inside the tube 15 being arranged on either side of the longitudinal axis of the tube, close to the walls of the tube.

Of course, a tube structure 6 can be provided adapted to allow bending in two planes perpendicular to each other using three or four actuation wires 13 in order to obtain the left-to-right and top-to-bottom movement of the distal head 4. According to this variant embodiment, the tube 15 is cut to have a third series of cut-out areas forming pivots of rotation and by a fourth series of cut-out areas forming pivots of rotation extending diametrically opposite the third series and along a plane perpendicular to the first plane of symmetry S, i.e., the diametrical bending plane D. This variant embodiment is not described more precisely because it follows directly from the preceding description.

According to the present disclosure, the bending structure 6 has a system 23 for retaining the tubular vertebrae together between the two opposite extreme positions likely to be taken during bending of the bending structure. In other words, this retaining system 23 makes it possible to keep the tubular vertebrae 16 engaged over the entire bending amplitude while limiting this bending in both ways of the direction perpendicular to the first axis of rotation X, in two opposite extreme positions.

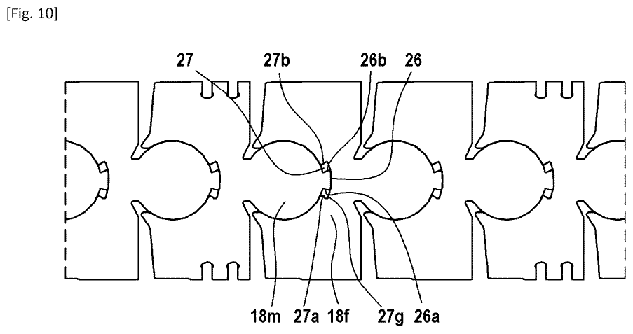

The retaining system 23 according to the present disclosure has at least one bulge or lug 26 extending projecting from the male cut-out areas 17m, 18m and having a first bearing surface 26a and a second bearing surface 26b. Each lug 26 thus has, opposite the male cut-out area, a free end called outer edge 26e. Each lug 26 is delimited on either side by the two rotation limiting surfaces 26a, 26b which extend projecting from the male cut-out areas 17m, 18m and up to the outer edge 26e of the lug. Each lug 26 is engaged in a slot 27 arranged in the female cut-out areas 17f, 18f and having a first and a second surface 27a, 27b for limiting rotation around the axis of rotation X. The first and second surfaces 27a, 27b for limiting rotation are located on either side of the plane of symmetry S to lock the bending of the bending structure 6 in two opposite extreme positions. According to the variant embodiment illustrated in FIG. 10, each male cut-out area 17m, 18m is provided with a single lug 26 engaged in a single slot 27 while according to the variant embodiment illustrated in FIGS. 2 to 9, each male cut-out area 17m, 18m is provided with two lugs 26 projecting from each of the male cut-out areas 17m, 18m and each engaged in a slot 27 of the female cut-out areas 17f, 18f and presenting a rotation limiting surface.

According to the variant embodiment illustrated in FIGS. 2 to 9, each male cut-out area 17m, 18m has a first lug 26 presenting the first bearing surface 26a and a second lug 26 presenting the second bearing surface 26b. These first and second bearing surfaces 26a and 26b are intended to come into contact, respectively, with the first bearing surface 27a for limiting rotation around the axis of rotation X and the second surface 27b for limiting rotation around the axis of rotation X. Thus, one of the lugs 26 of a male cut-out area 17m, 18m is intended to come into contact by its first bearing surface 26a, with the first surface 27a for limiting rotation around the axis of rotation X, during rotation in a first direction while the other lug 26 of the male cut-out area 17m, 18m is intended to come into contact through its second bearing surface 26b, with the second surface 27b for limiting the rotation around the axis of rotation X, during rotation in a second direction opposite the first direction of rotation.

According to the variant embodiment illustrated in FIG. 10, each lug 26 of a male cut-out area 17m, 18m has two bearing surfaces 26a, 26b arranged on either side of the lug which is engaged in a slot 27 delimited on either side by the first and second rotation limiting surfaces 27a, 27b. The first bearing surface 26a of these lugs is intended to come into contact with the first surface 27a for limiting rotation around the axis of rotation X during rotation in a first direction while the second bearing surface 26b, is intended to come into contact with the second surface 27b for limiting rotation around the axis of rotation X, during rotation in a second direction opposite the first direction of rotation.

It appears from the preceding description that for the lug(s) 26 of each male cut-out area 17m, 18m, the two rotation limiting surfaces 27a, 27b are located on either side of the plane of symmetry S so that one of the limiting surfaces blocks rotation in one direction while the other limiting surface blocks rotation in the other direction. In other words, the bending of the bending structure 6 is limited in the diametrical bending plane D in both ways of the direction perpendicular to the first axis of rotation X, such as, for example, top-to-bottom or left-to-right.

According to an advantageous characteristic, for each extreme position between two neighbouring tubular vertebrae, the two lugs 26 of the two diametrically opposed male cut-out areas 17m, 18m are supported on the limiting surfaces 27a or 27b of slots 27 located on one side of the plane of symmetry S, this side of the plane being located opposite the direction of bending. By convention, the side of the plane of symmetry S located opposite the direction of bending is called the outer side while the side of the plane of symmetry S located on the side of the direction of bending, is called the inner side. In the example illustrated in FIGS. 3 to 5, the bending direction of the bending structure 6 is the right side (inner side) of bending so that the part of the first outer edges 16a of the tubular vertebrae delimited by the first series of notches 20 abut in the so-called right extreme bending position, against the part of the second outer edges 16b of the neighbouring tubular vertebrae 16 delimited by this first series of notches 20. In the so-called right extreme bending position, the two lugs 26 of two diametrically opposed male cut-out areas 17m, 18m of the same tubular vertebra are supported by their first bearing surfaces 26a, on the first limiting surfaces 27a of the of the slots 27 located on the left or outer side of the plane of symmetry S (FIG. 4).

Of course, when the bending direction of the bending structure 6 is the left side of bending, the part of the first outer edges 16a of the tubular vertebrae delimited by the second series of notches 21 abut in the so-called left extreme bending position, against the part of the second outer edges 16b of the neighbouring tubular vertebrae 16 delimited by this second series of notches 21. In the so-called left extreme bending position, the two lugs 26 of two diametrically opposed male cut-out areas 17m, 18m of the same tubular vertebra are supported on the second limiting surfaces 27b of the slots 27 located on the right or outer side of the plane of symmetry S.

Each slot 27 is advantageously arranged in a female cut-out area 17f, 18f according to an annular sector centred on the axis of rotation X. Thus, each slot 27 is delimited by a guide edge 27g centred on the axis of rotation to serve as a guide for the outer edge 26e of the lug 26. Thus, each lug 26 cooperates via its outer edge 26e with the guide edge 27g, thus completing the effect of the rotation pivots.

According to an advantageous embodiment, the lugs 26 have a first bearing surface 26a and a second bearing surface 26b not passing through the axis of rotation X. This first bearing surface 26a and this second bearing surface 26b are intended to cooperate in an extreme position, respectively with a rotation limiting surface 27a, 27b extending along a plane N not passing through the axis of rotation X (FIG. 5). Such an arrangement allows the lug 26 and the female cut-out area 17f, 18f to nest one inside the other, helping to keep them anchored.

According to another characteristic of the present disclosure, the retaining system 23 according to the present disclosure also has a pair of hooks, i.e., a first hook 30 and a second hook 31, extending projecting from the tubular vertebrae 16, outside the diametrical bending plane D and in orthogonal symmetry relative to the longitudinal axis L. A tubular vertebra 16 is thus provided with a first hook 30 and a second hook 31 forming a pair. Each hook 30, 31 of a pair belonging to a tubular vertebra is engaged in a housing, respectively a first housing 32 and a second housing 33, arranged in a neighbouring tubular vertebra 16. Each housing 32, 33 has a retaining stop 34 on which the hook comes to rest in one of the two extreme positions as defined previously. More precisely, the hook of a pair 30, 31 comes to rest against a retaining stop 34 in one of the two extreme positions while the other hook of the pair 30, 31 comes to rest against a retaining stop 34 in the other of the two extreme positions, as will be described in detail later in the description. In the illustrated embodiment, the hooks 30, 31 extend in the extension of the tubular vertebrae, projecting from the first outer edge 16a of the tubular vertebrae while the recesses 32, 33 are arranged from the second outer edge 16b of the tubular vertebrae.

According to one characteristic of the present disclosure, each hook 30, 31 has an L shape with a rectilinear shank 36 of constant segment, curved at its end, for example at a right angle on one side while remaining in the plane of extension of the rectilinear shank (FIG. 7). For each hook 30, 31, the rectilinear shank 36 has at its free end, a heel 37 delimiting, opposite the end 37a of the shank, a retaining surface 37b extending in the example illustrated perpendicular to the rectilinear shank 36. It should be noted that the heel can be curved beyond a right angle so that the retaining surface 37b forms with the rectilinear shank, an acute angle favouring hooking. As can be seen from the drawings, for each hook 30, 31, the end 37a of the shank has, for example, a wall in the shape of an arc of a circle while the retaining surface 37b has a flat wall.

For each pair of hooks 30, 31 equipping a tubular vertebra 16, the first hook 30 is located along a first side of the plane of symmetry S while the second hook 31 is located along a second side of the plane of symmetry S, opposite the first side. For one of the two extreme positions of the tubular vertebrae, the first hook 30 is supported by its retaining surface 37b, on the retaining stop 34 of the first housing 32 while for the other of the extreme positions, the second hook 31 is supported by its retaining surface 37b, on the retaining stop 34 of the second housing 33. Preferably, the retaining surface 37b and the retaining stop 34 have complementary or congruent surfaces.

As a reminder, in the example illustrated in FIGS. 3 to 5, the bending direction of the bending structure 6 is the right side (inner side of the curve) so that the part of the first outer edges 16a of the tubular vertebrae delimited by the first series of notches 20 abut, in the so-called right extreme bending position, against the part of the second outer edges 16b of the neighbouring tubular vertebrae 16 delimited by this first series of notches 20. In the so-called right extreme bending position, the first hooks 30 located relative to the plane of symmetry S, on the side located opposite the bending direction are supported on the retaining stop 34 (FIG. 4). In other words, the first hooks 30 located on the outer side are supported by the retaining surface 37b, on the retaining stop 34. Conversely, in the so-called left extreme bending position, the second hooks 31 located relative to the plane of symmetry S, on the side located opposite the bending direction are supported on the retaining stop 34. The second hooks 31 located on the outer side are supported on the retaining stop 34, by the retaining surface 37b.

It appears from the preceding description that the retaining system 23 is arranged in such a way that for each extreme position between two neighbouring tubular vertebrae, on the one hand, the two lugs 26 of two male cut-out areas 17m, 18m diametrically opposite one another of a tubular vertebra are supported on the limiting surfaces 27a, 27b of the slots located on one side of the plane of symmetry S located opposite the bending direction and, on the other hand, the hook 30, 31 located on the side of this plane of symmetry, that is, supported on the retaining stop 34. It therefore appears that for each extreme position, the retaining system 23 implements, for two neighbouring tubular vertebrae 16, three additional contact points located on the outer side of the bending structure, thus preventing the tubular vertebrae from disengaging from each other.

Each housing 32, 33 is opened in the second outer edge 16b, respectively, by a narrowed part 39 for guiding the rectilinear shank 36 of the hooks. The heel 37 of each hook is engaged in a housing 32, 33 delimited on the one hand, on the side farthest from the second outer edge 16b of the tubular vertebra, by a bottom 40 of the recesses 32, 33, in the shape of an arc of circle and on the other hand, on the side closest to the second outer edge 16b, by the retaining stop 34 connecting to the outlet of the narrowed part 39. Each housing 32, 33 is also delimited between the bottom 40 and the retaining stop 34, by two guiding edges 41 for the heel as it slides. Thus, each housing 32, 33 has a substantially rectangular outline extending by the narrowed part 39 also of rectangular shape.

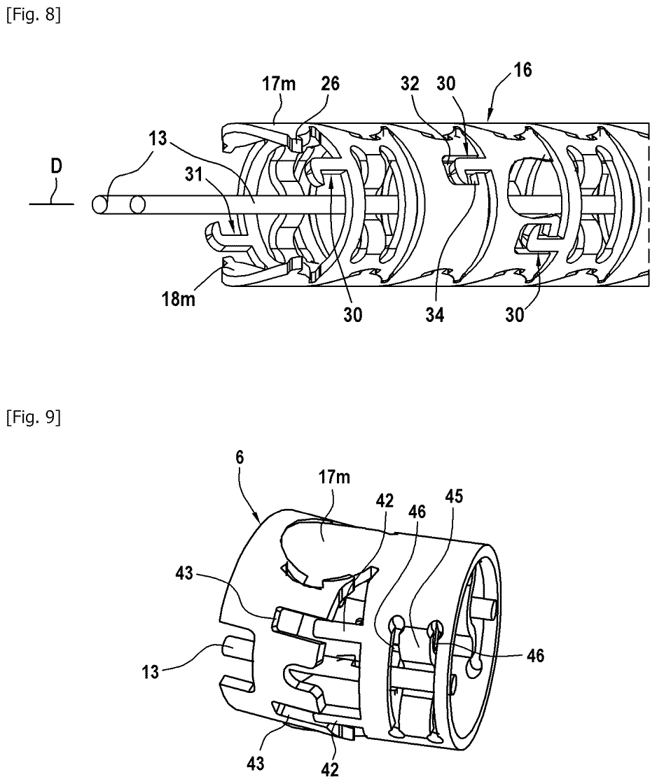

In accordance with one characteristic of the present disclosure, the hooks 30, 31 of a pair extend from each tubular vertebra, in orthogonal symmetry relative to the longitudinal axis L. Thus, as appears, for example, from FIG. 8, the two hooks 30, 31 of a pair are identical in shape and dimensions but the heels 37 are oriented in a diametrically opposite way. For example, the heel 37 of the first hook 30 extending from the extreme tubular vertebra of FIG. 8 is oriented towards the bottom of the figure while the heel 37 of the second hook 31 extending from the extreme tubular vertebra of FIG. 8 is oriented towards the top of the figure. The heels 37 of the hooks 30, 31 of a pair adjoining a tubular vertebra are oriented in the same direction, in a direction of rotation around the longitudinal axis L.

This pair of hooks 30, 31 is located symmetrically on either side of the longitudinal axis L, being positioned outside the diametrical bending plane D along which the actuation wires 13 are centred. The hooks 30, 31 are not located in the diametrical bending plane D to be thus separated from the actuation wires 13 so as not to interfere in the operation of the actuation wires 13. Thus, as appears in the figures, for each pair of hooks 30, 31 associated with a tubular vertebra, one of the hooks 30 is positioned on one side of the plane of symmetry S while the other hook 31 is positioned on the other side of the plane of symmetry S.

It should be noted that one of the hooks of a pair is positioned on one side of the diametrical bending plane D while the other hook of said pair is positioned on the other side of the diametrical bending plane D. According to an advantageous embodiment, the hooks 30, 31 are arranged from one tubular vertebra to the other alternately on either side of the diametrical bending plane D. Thus, as appears, for example, from FIG. 4, for the hooks 30, 31 located on one side of the plane of symmetry S (the one visible in the drawing), the hooks 30 are alternately, from one tubular vertebra 16 to the other, located below and above the diametrical bending plane D. It should be noted that the orientation of the heels 37 of the hooks changes direction from one tubular vertebra to another. For example, for the hooks 30 located on the side of the plane of symmetry S visible in FIG. 4, the heels 37 of the hooks located above the diametrical bending plane D are oriented towards the bottom of the figure while the heels 37 of the hooks located below the diametrical bending plane D are oriented towards the top of the figure.

Likewise, for the hooks located on the other side of the plane of symmetry S (the one not visible in FIG. 4 but visible through the cut-out parts of FIG. 7), the hooks 31 are alternately, from one tubular vertebra to the other, located, respectively, above and below the diametrical bending plane D. This crossed distribution of the hooks 30, 31 makes it possible to distribute the areas retaining the tubular vertebrae among them, helping to retain the tubular vertebrae in different directions during the application of forces on tube 15.

According to an advantageous variant embodiment, the retaining system 23 has, for at least one tubular vertebra 16, two pairs of tongues 42 arranged symmetrically on either side of the plane of symmetry S (FIG. 9). Thus, one pair of tongues 42 is arranged on one side of the plane of symmetry S while the other pair of tongues 42 is arranged on the other side of the plane of symmetry S. Each tongue 42 has a rectilinear shape made parallel to the longitudinal axis L of the tube 15 and intended to cooperate with a groove 43 of complementary shape. The tongues 42 extend projecting from an outer edge of a tubular vertebra, outside the diametrical bending plane D, each being engaged in a groove 43 made in the neighbouring tubular vertebra. The tongues 42 and the grooves 43 are thus arranged so as not to interfere with the actuation wires 13. For example, the two pairs of tongues 42 are arranged on the proximal tubular vertebra 16p and/or on the distal tubular vertebra 16d and/or on tubular vertebrae not having the hooks 30, 31.

Advantageously, to hold the actuation wires 13 in position inside the tube 15 (FIG. 9), some tubular vertebrae 16 have a retaining tongue 45 cut from the tube by two parallel cutting lines 46 while remaining attached at its ends. This retaining tongue 45 is pushed back inside the tube 15 to constitute a guide eye for the actuation wire 13. Of course, as illustrated in the drawings, the retaining tongues 45 can be arranged in tubular vertebrae located between the proximal tubular vertebra and the distal tubular vertebra and not having a retaining system 23 according to the present disclosure.

1. Bending structure for an insertion tube of a medical device, having tubular vertebrae with a proximal tubular vertebra and a distal tubular vertebra and at least one actuation wire extending in a diametrical bending plane of the structure, the bending structure having a tube with a longitudinal axis, cut by cutting lines of an energy beam to produce tubular vertebrae nested in one another by diametrically opposed male cut-out areas and diametrically opposed female cut-out areas forming pivots of rotation along axes of rotation located in a plane of symmetry, the cutting lines being arranged to delimit, between the tubular vertebrae, notches cut in the tube to form bending zones for the tubular vertebrae in order to obtain the bending of the bending structure in the diametrical bending plane perpendicular to the axes of rotation, the bending structure having a system for retaining the tubular vertebrae together between two opposite extreme positions, the retaining system having:
at least one lug extending projecting from the male cut-out areas and engaged in a slot in the female cut-out areas and having two rotation limiting surfaces located on either side of the plane of symmetry,
a pair of hooks extending projecting from the tubular vertebrae, outside the diametrical bending plane and in orthogonal symmetry relative to the longitudinal axis, each hook having an L shape with a rectilinear shank curved at its end, on one side to present a heel delimiting, opposite the end of the shank, a retaining surface, the hooks each being engaged in a housing arranged in a neighbouring tubular vertebra opening in an outer edge of said neighbouring tubular vertebra by a narrowed part for guiding the rectilinear shank bordered by a retaining stop on which the hook comes to rest by its heel in the two extreme positions.

2. A bending structure according to claim 1, wherein the hooks are arranged from one tubular vertebra to the other alternately on either side of the diametrical bending plane.

3. The bending structure according to claim 1, wherein the retaining surface has a flat wall.

4. The bending structure according to claim 1, wherein the retaining system is arranged in such that for each extreme position between two neighbouring tubular vertebrae, the two lugs of two male cut-out areas diametrically opposed are supported on the rotation limiting surfaces of the slots located on one side of the plane of symmetry located opposite the bending direction and the hook located on the side of this plane of symmetry is supported on the retaining stop.

5. The bending structure according to claim 4, wherein the two lugs extend projecting from each of the male cut-out areas and are each engaged in a slot in the female cut-out areas and have a first bearing surface and a second bearing surface intended to cooperate with a rotation limiting surface.

6. The bending structure according to claim 4, wherein each lug has at least one bearing surface not passing through the axis of rotation and intended to cooperate in an extreme position, with a rotation limiting surface extending along a plane not passing through the axis of rotation, so as to nest one inside the other.

7. The bending structure according to claim 6, wherein the at least one bearing surfaces of each lug extends projecting from the male cut-out areas and up to the outer edge of the lug.

8. The bending structure according to claim 1, wherein each male cut-out area has a connecting neck with the part forming a rotation pivot.

9. The bending structure according to claim 1, wherein each female cut-out area is arranged to connect with an outer edge of the tubular vertebra, using non-pointed reinforcing heels extending on either side of the part forming a rotation pivot and configured to engage in the connecting neck.

10. The bending structure according to claim 1, wherein the retaining system has, for at least one tubular vertebra, two pairs of tongues arranged symmetrically on either side and outside the diametrical bending plane, each being engaged in a groove provided in a neighbouring tubular vertebra.

11. The bending structure according to claim 1, wherein the tube has a diameter less than or equal to 10 mm.

12. The bending structure according to claim 1, wherein the tube has a wall thickness comprised between 0.05 and 1.5 mm.

13. The bending structure according to claim 1, wherein the tube for insertion is an endoscope or catheter fixed to the tube at its proximal end and to a distal head at its distal end.

14. The bending structure according to claim 1, wherein the bending structure includes an operating handle equipped at its distal part.
